(19) United States
(12) Patent Application Publication
(10) Pub. No.: US 2011/0293515 A1
(43) Pub. Date: Dec. 1, 2011

(54) HETEROFUNCTIONAL SEGMENT-POLY (ETHYLENE GLYCOL) POLYMERS AS DELIVERY VEHICLES

(75) Inventors: Ahmad Safavy, Birmingham, AL (US); James A. Bonner, Birmingham, AL (US); Donald J. Buchsbaum, Alabaster, AL (US)

(73) Assignee: THE UAB RESEARCH FOUNDATION, Birmingham, AL (US)

(21) Appl. No.: 13/144,440

(22) PCT Filed: Jan. 12, 2010
(86) PCT No.: PCT/US10/20739

§ 371 Date: Aug. 11, 2011

Related U.S. Application Data
(60) Provisional application No. 61/144,221, filed on Jan. 13, 2009.

(51) Int. Cl. A61K 51/06; A61P 35/00; A61K 49/00; C08G 69/48; A61K 47/48

(52) U.S. Cl. 424/1.65; 525/436; 525/54.1; 525/54.2; 424/78.17; 424/9.1

(57) ABSTRACT

Heterofunctional segment-poly(ethylene glycol) polymers, conjugates of these molecules with therapeutic and/or imaging agents, and methods for their use are disclosed. The heterofunctional segment-poly(ethylene glycol) polymers are useful as drug delivery conjugates, i.e., the heterofunctional segment-poly(ethylene glycol) polymers can be covalently attached to therapeutic agents (e.g., pharmaceutically active agents) and serve as delivery vehicles for the therapeutic agents. The heterofunctional segment-poly(ethylene glycol) polymers are also useful as imaging agent conjugates, i.e. the heterofunctional segment-poly(ethylene glycol) polymers can be covalently attached to imaging agents (e.g., tracers, imaging atoms, and imaging molecules) and serve as delivery vehicles for the imaging agents. Also disclosed are methods for treating a subject by administering to the subject an effective amount of the polymers conjugated to a therapeutic agent, an imaging agent, or a mixture thereof.

### CROSS-REFERENCE TO PRIORITY APPLICATIONS

This application claims priority to U.S. Provisional Application No. 61/144,221, filed Jan. 13, 2009, which is incorporated herein by reference in its entirety.

### BACKGROUND

Polymers can be used to deliver pharmaceutically active agents and imaging agents in biological systems. Such polymeric delivery molecules can induce aqueous solubility to natively insoluble pharmaceutically active agents and imaging agents. The use of such polymers can also increase the circulation time of pharmaceutically active agents or imaging agents, thus improving target localization, which can also improve the specificity of an agent or aid in improving the accuracy of clinical diagnosis.

### SUMMARY

Heterofunctional segment-poly(ethylene glycol) polymers, conjugates of these compounds with therapeutic agents and/or imaging agents, and methods for their use are disclosed. A class of polymers comprises compounds of the following formula:

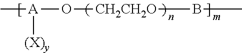

and salts thereof. In this class of compounds, A is substituted or unsubstituted C₁₋₁₂ alkyl, substituted or unsubstituted C₂₋₁₂ alkenyl, substituted or unsubstituted C₂₋₁₂ alkynyl, substituted or unsubstituted C₂₋₁₂ heteroalkyl, substituted or unsubstituted C₂₋₁₂ heteroalkenyl, or substituted or unsubstituted C₂₋₁₂ heteroalkynyl; B is substituted or unsubstituted C₁₋₁₂ alkyl, substituted or unsubstituted C₂₋₁₂ alkenyl, substituted or unsubstituted C₂₋₁₂ alkynyl, substituted or unsubstituted C₂₋₁₂ heteroalkyl, substituted or unsubstituted C₂₋₁₂ heteroalkenyl, substituted or unsubstituted C₂₋₁₂ heteroalkynyl, or is absent; m, n, and y are each independently selected from an integer of one or greater; and X is a linker molecule covalently attached to A.

Also disclosed is a method of treating a subject, comprising administering to the subject an effective amount of a compound of the following formula:

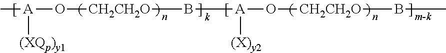

and pharmaceutically acceptable salts thereof. In this class of compounds, A is substituted or unsubstituted C₁₋₁₂ alkyl, substituted or unsubstituted C₂₋₁₂ alkenyl, substituted or unsubstituted C₂₋₁₂ alkynyl, substituted or unsubstituted C₂₋₁₂ heteroalkyl, substituted or unsubstituted C₂₋₁₂ heteroalkenyl, or substituted or unsubstituted C₂₋₁₂ heteroalkynyl; B is substituted or unsubstituted C₁₋₁₂ alkyl, substituted or unsubstituted C₂₋₁₂ alkenyl, substituted or unsubstituted C₂₋₁₂ alkynyl, substituted or unsubstituted C₂₋₁₂ heteroalkyl, substituted or unsubstituted C₂₋₁₂ heteroalkenyl, substituted or unsubstituted C₂₋₁₂ heteroalkynyl, or is absent; m, n, p, y¹, and y² are each independently selected from an integer of one or greater; k is an integer from one to m; Q is a therapeutic agent; and X is a linker molecule covalently attached to A.

Also disclosed is a method of detecting specific target cells in a subject, comprising administering to the subject an effective amount of a compound of the following formula:

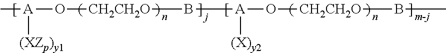

and pharmaceutically acceptable salts thereof, and detecting localization of the compound by a diagnostic imaging technique. In this class of compounds, A is substituted or unsubstituted C₁₋₁₂ alkyl, substituted or unsubstituted C₂₋₁₂ alkenyl, substituted or unsubstituted C₂₋₁₂ alkynyl, substituted or unsubstituted C₂₋₁₂ heteroalkyl, substituted or unsubstituted C₂₋₁₂ heteroalkenyl, or substituted or unsubstituted C₂₋₁₂ heteroalkynyl; B is substituted or unsubstituted C₁₋₁₂ alkyl, substituted or unsubstituted C₂₋₁₂ alkenyl, substituted or unsubstituted C₂₋₁₂ alkynyl, substituted or unsubstituted C₂₋₁₂ heteroalkyl, substituted or unsubstituted C₂₋₁₂ heteroalkenyl, substituted or unsubstituted C₂₋₁₂ heteroalkynyl, or is absent; m, n, p, y¹, and y² are each independently selected from an integer of one or greater; j is an integer from one to m; Z is an imaging agent; and X is a linker molecule covalently attached to A.

Further disclosed is a method of treating a subject and detecting specific target cells in a subject, comprising administering to the subject an effective amount of a compound of the following formula:

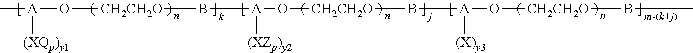

and pharmaceutically acceptable salts thereof, and detecting localization of the compound by a diagnostic imaging technique. In this class of compounds, A is substituted or unsubstituted C₁₋₁₂ alkyl, substituted or unsubstituted C₂₋₁₂ alkenyl, substituted or unsubstituted C₂₋₁₂ alkynyl, substituted or unsubstituted C₂₋₁₂ heteroalkyl, substituted or unsubstituted C₂₋₁₂ heteroalkenyl, or substituted or unsubstituted C₂₋₁₂ heteroalkynyl; B is substituted or unsubstituted C₁₋₁₂ alkyl, substituted or unsubstituted C₂₋₁₂ alkenyl, substituted or unsubstituted C₂₋₁₂ alkynyl, substituted or unsubstituted C₂₋₁₂ heteroalkyl, substituted or unsubstituted C₂₋₁₂ heteroalkenyl, substituted or unsubstituted C₂₋₁₂ heteroalkynyl, or is absent; m, n, p, y¹, y², and y³ are each independently selected from an integer of one or greater; j and k are each independently selected from an integer from one to m; Q is a therapeutic agent; Z is an imaging agent; and X is a linker molecule covalently attached to A.

Also described are methods of making these compounds, comprising conjugating a therapeutic agent, an imaging agent, or mixtures thereof to a heterofunctional segment-poly(ethylene glycol) polymer using a coupling reagent.

### DESCRIPTION OF DRAWINGS

FIG. 1 is a bar graph showing the cytotoxicity of unconjugated camptothecin (CPT-10-OH) and conjugated camptothecin (AS-X-147) in a human colorectal cancer cell line.

FIG. 2 is a bar graph showing the cytotoxicity of unconjugated camptothecin (CPT-10-OH) in a human colorectal cancer cell line.

FIG. 3 is a bar graph showing the cytotoxicity of conjugated camptothecin (AS-X-147) in a human colorectal cancer cell line.

FIG. 4 is a bar graph showing the cytotoxicity of unconjugated paclitaxel (Taxol) and conjugated paclitaxel (AS-X-146) in a human prostate cancer cell line.

### DETAILED DESCRIPTION

Heterofunctional segment-poly(ethylene glycol) polymers, conjugates of these molecules with therapeutic agents and/or imaging agents, and methods for their use are disclosed. The polymers consist of a heterofunctional segment and a poly(ethylene glycol) segment that alternate throughout the polymeric chain in a head-to-tail arrangement. The heterofunctional segment-poly(ethylene glycol) polymers are useful as drug delivery conjugates, i.e., the heterofunctional segment-polyethylene glycol polymers can be covalently attached to therapeutic agents and serve as delivery vehicles for the therapeutic agents. The heterofunctional segment-poly(ethylene glycol) polymers are also useful as imaging agent conjugates, i.e. the heterofunctional segment-poly(ethylene glycol) polymers can be covalently attached to imaging agents (e.g., tracers, imaging atoms, and imaging molecules) and serve as delivery vehicles for the imaging agents.

The heterofunctional segment-poly(ethylene glycol) polymers described herein are represented by Compound I:

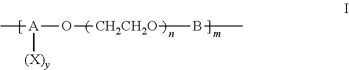

or a salt thereof.

In Compound I, A is substituted or unsubstituted C₁₋₁₂ alkyl, substituted or unsubstituted C₂₋₁₂ alkenyl, substituted or unsubstituted C₂₋₁₂ alkynyl, substituted or unsubstituted C₂₋₁₂ heteroalkyl, substituted or unsubstituted C₂₋₁₂ heteroalkenyl, or substituted or unsubstituted C₂₋₁₂ heteroalkynyl. In one example, A is

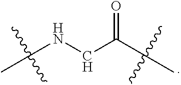

Also in Compound I, B is substituted or unsubstituted C₁₋₁₂ alkyl, substituted or unsubstituted C₂₋₁₂ alkenyl, substituted or unsubstituted C₂₋₁₂ alkynyl, substituted or unsubstituted C₂₋₁₂ heteroalkyl, substituted or unsubstituted C₂₋₁₂ heteroalkenyl, substituted or unsubstituted C₂₋₁₂ heteroalkynyl, or is absent. In some examples, B is

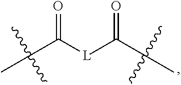

wherein L is a substituted or unsubstituted C₁₋₁₂ alkyl. For example, L can be —CH₂—CH₂—.

Additionally in Compound I, m, n, and y are each independently selected from an integer of one or greater. In one example, m is 45, n is 6, and y is 1.

Further, in Compound I, X is a linker molecule covalently attached to A. In some examples, X is substituted or unsubstituted C₁₋₁₂ alkyl, substituted or unsubstituted C₂₋₁₂ alkenyl, substituted or unsubstituted C₂₋₁₂ alkynyl, substituted or unsubstituted C₂₋₁₂ heteroalkyl, substituted or unsubstituted C₂₋₁₂ heteroalkenyl, or substituted or unsubstituted C₂₋₁₂ heteroalkynyl.

As used herein, the terms alkyl, alkenyl, and alkynyl include straight- and branched-chain monovalent substituents. Examples include methyl, ethyl, isobutyl, 3-butynyl, and the like. Heteroalkyl, heteroalkenyl, and heteroalkynyl are similarly defined but may contain O, S, or N heteroatoms or combinations thereof within the backbone.

The alkyl, alkenyl, alkynyl, heteroalkyl, heteroalkenyl, and heteroalkynyl molecules used herein can be substituted or unsubstituted. As used herein, the term substituted includes the addition of an alkyl, alkenyl, alkynyl, aryl, heteroalkyl, heteroalkenyl, heteroalkynyl, or heteroaryl group to a position attached to the main chain of the alkyl, alkenyl, alkynyl, heteroalkyl, heteroalkenyl, or heteroalkynyl, e.g., the replacement of a hydrogen by one of these molecules. Examples of substitution groups include, but are not limited to, hydroxyl, halogen (e.g., F, Br, Cl, or I), and carboxyl groups. Conversely, as used herein, the term unsubstituted indicates the alkyl, alkenyl, alkynyl, heteroalkyl, heteroalkenyl, or heteroalkynyl has a full complement of hydrogens, i.e., commensurate with its saturation level, with no substitutions, e.g., linear decane (—(CH₂)₉—CH₃).

Aryl molecules include, for example, cyclic hydrocarbons that incorporate one or more planar sets of, typically, six carbon atoms that are connected by delocalized electrons numbering the same as if they consisted of alternating single and double covalent bonds. An example of an aryl molecule is benzene. Heteroaryl molecules include substitutions along their main cyclic chain of atoms such as O, N, or S. When heteroatoms are introduced, a set of five atoms, e.g., four carbon and a heteroatom, can create an aromatic system. Examples of heteroaryl molecules include furan, pyrrole, thiophene, imidazole, oxazole, pyridine, and pyrazine. Aryl and heteroaryl molecules can also include additional fused rings, for example, benzofuran, indole, benzothiophene, naphthalene, anthracene, and quinoline.

In certain examples of Compound I, it is useful to consider A and X as a single unit. A and X taken together (i.e., A-X) can be, for example, an amino acid. The side chains of acidic and basic amino acids can be protonated or deprotonated. For example, if A is

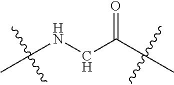

and X is —(CH₂)₂CO₂H, then A-X is glutamic acid. An example of Compound I where A-X is glutamic acid is as follows:

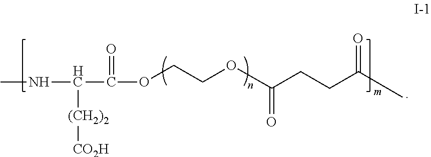

Other naturally and non-naturally occurring amino acids, e.g., arginine; asparagine; aspartic acid; citrulline; cysteine; glutamine; histidine; lanthionine; lysine; ornithine; serine; threonine; tryptophan; and tyrosine, are also useful.

Also in Compound I, m can be an integer of 1 or greater. For example, m can be 1 or greater, 2 or greater, 3 or greater, 4 or greater, 5 or greater, 6 or greater, 7 or greater, 8 or greater, 9 or greater, 10 or greater, 15 or greater, 20 or greater, 25 or greater, 30 or greater, 40 or greater, 50 or greater, 60 or greater, 70 or greater, 80 or greater, 90 or greater, 100 or greater, 125 or greater, 150 or greater, 175 or greater, 200 or greater, 225 or greater, 250 or greater, 275 or greater, 300 or greater, 400 or greater, 500 or greater, 600 or greater, 700 or greater, 800 or greater, 900 or greater, or 1000 or greater. Useful ranges for m include from 1 to 1000, from 1 to 100, from 1 to 10, or any subranges thereof.

Additionally in Compound I, n can be an integer of 1 or greater. For example, n can be 1 or greater, 2 or greater, 3 or greater, 4 or greater, 5 or greater, 6 or greater, 7 or greater, 8 or greater, 9 or greater, 10 or greater, 15 or greater, 20 or greater, 25 or greater, 30 or greater, 40 or greater, 50 or greater, 60 or greater, 70 or greater, 80 or greater, 90 or greater, or 100 or greater. Useful ranges for n include from 1 to 100, from 1 to 50, from 1 to 10, or any subranges thereof.

Further in Compound I, y can be an integer of 1 or greater. For example, y can be 1 or greater, 2 or greater, 3 or greater, 4 or greater, 5 or greater, 6 or greater, 7 or greater, 8 or greater, 9 or greater, or 10 or greater. Useful values for y include 1, 2, 3, 4, 5, 6, 7, 8, 9, and 10.

A further example of Compound I includes:

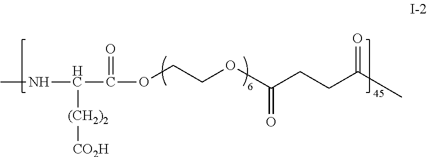

Additionally in Compound I, one or more of a therapeutic agent, an imaging agent, or mixtures thereof can be attached to X through a covalent bond to the main chain of X or through a covalent bond to a substitution group of X. Examples of Compound I with a therapeutic agent attached to X include:

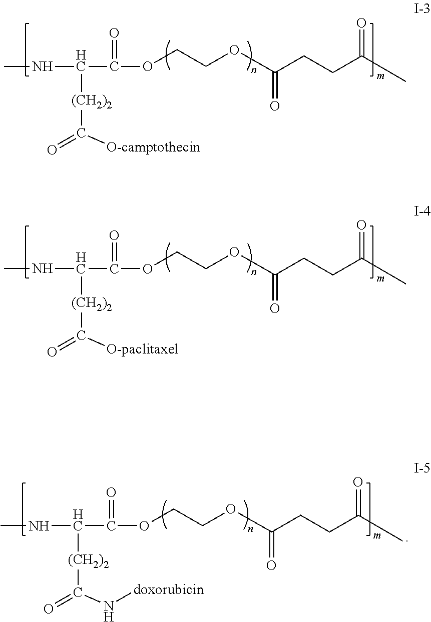

As used herein, the term therapeutic agent is intended to mean an agent or drug that when provided to a subject in an effective amount will have a desired effect. Examples of therapeutic agents useful with the compounds and methods described herein include anti-cancer agents, radioactive isotopes, polypeptides, and carbohydrates. Anti-cancer agents include, for example, antibodies, anthracyclines, bleomycin, calicheamicins, camptothecin, carboplatin, chlorambucil, cisplatin, colchicine, curcumin, daunorubicin, dactinomycin, diethylstilbestrol, doxorubicin, dynemicines, esperamicins, etoposide, 5-fluorouracil, floxuridine, FR-900482/FR-66979, melphalan, 6-mercaptopurine, methotrexate, mitomycin, nitrogen mustards, paclitaxel, platinum derived agents, teniposide, 6-thioguanine, vincristine and vinblastine, or derivatives thereof. Further examples of anti-cancer agents and therapeutic agents are found in The Merck Manual of Diagnosis and Therapy, 18th Ed., Beers et al., eds., 2006, Whitehouse Station, N.J.; Anticancer Drugs: Reactive Metabolism and Drug Interactions, 1994, 1^{st} Ed., Powis, G., ed., Pergamon Press, Oxford, England; and Pratt et al. The Anticancer Drugs, 2^{nd} Ed., 1994, Oxford University Press, New York, N.Y. As used herein, the terms polypeptide and peptide are used interchangeably, and refer to an amino acid sequence of any length.

As used herein, the term imaging agent is intended to mean any compound or combination of compounds that enhance the visualization and differentiation of cells, organs, and other cellular structures from the surrounding medium. Examples of imaging agents useful with the compounds and methods described herein include tracers, imaging atoms, and imaging molecules.

Therapeutic agents or imaging agents may be attached to one or more of the heterofunctional segment-poly(ethylene glycol) polymer units. For example, for a heterofunctional segment-poly(ethylene glycol) polymer with 45 units, the therapeutic agents could be attached to from one to 45 of the units. In addition, the heterofunctional segment-poly(ethylene glycol) polymer may contain mixtures of both therapeutic agents and imaging agents. The percentage of therapeutic agent conjugated units or imaging agent conjugated units within the heterofunctional segment-poly(ethylene glycol) polymer may be varied (e.g., from less than 1% to 100% or any amount in between) in order to produce the desired effect in the subject.

Also described is a method of treating a subject, comprising administering to the subject an effective amount of a compound as represented by Compound II:

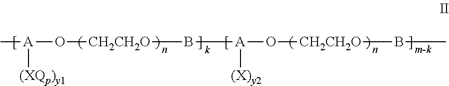

or a pharmaceutically acceptable salt thereof.

In Compound II, A is substituted or unsubstituted C₁₋₁₂ alkyl, substituted or unsubstituted C₂₋₁₂ alkenyl, substituted or unsubstituted C₂₋₁₂ alkynyl, substituted or unsubstituted C₂₋₁₂ heteroalkyl, substituted or unsubstituted C₂₋₁₂ heteroalkenyl, or substituted or unsubstituted C₂₋₁₂ heteroalkynyl. In one example, A is

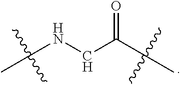

Also in Compound II, B is substituted or unsubstituted C₁₋₁₂ alkyl, substituted or unsubstituted C₂₋₁₂ alkenyl, substituted or unsubstituted C₂₋₁₂ alkynyl, substituted or unsubstituted C₂₋₁₂ heteroalkyl, substituted or unsubstituted C₂₋₁₂ heteroalkenyl, substituted or unsubstituted C₂₋₁₂ heteroalkynyl, or is absent. In some examples, B is

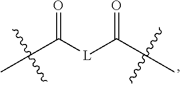

wherein L is a substituted or unsubstituted C₁₋₁₂ alkyl. For example, L can be —CH₂—CH₂—.

Also in Compound II, m, n, p, y¹, and y² are each independently selected from an integer of one or greater and k is an integer from one to m. As used in Compound II, the overall number of repeat units, i.e., the heterofunctional segment-poly(ethylene glycol) polymer units, is m, and the number of heterofunctional segment-poly(ethylene glycol) polymer units with an attached Q is k, thus, the number of heterofunctional segment-poly(ethylene glycol) polymer units with no Q is m−k. In one example, m is 45, n is 6, p is 1, y¹ is 1, y² is 1, and k is m (i.e., each heterofunctional segment-poly(ethylene glycol) polymer unit has an attached Q).

Additionally in Compound II, Q is a therapeutic agent as described herein. The k and m−k sections of Compound II are included because a therapeutic agent does not necessarily need to be attached to each repeat unit of Compound II for the compound to be effective. Thus, the amount of Q administered to a subject using Compound II will depend on the amount of the therapeutic agent (Q) delivered by Compound II to the area of interest and subsequently how much Q is available in the area of interest.

The orientation of the k and m−k sections of Compound II is not intended to indicate the Q bound subunits are necessarily in a block, but rather that a varying number of Q bound units, i.e., k, is possible. Expressed as a percentage, k can be as little as 1-2% of the total m units or as much as 100% (e.g., m=k). For example, k can be from 1% to 99%, from 5% to 95%, from 10% to 90%, from 20% to 80%, from 30% to 70%, or from 40% to 60% of m. Q can be, for example, paclitaxel, camptothecin, doxorubicin, curcumin, an antibody, or a platinum-derived agent. Examples of therapeutic agents useful with the compounds and methods as described herein include anti-cancer agents, radioactive isotopes, polypeptides, and carbohydrates.

Further in Compound II, X is a linker molecule covalently attached to A. In some examples, X is substituted or unsubstituted C₁₋₁₂ alkyl, substituted or unsubstituted C₂₋₁₂ alkenyl, substituted or unsubstituted C₂₋₁₂ alkynyl, substituted or unsubstituted C₂₋₁₂ heteroalkyl, substituted or unsubstituted C₂₋₁₂ heteroalkenyl, or substituted or unsubstituted C₂₋₁₂ heteroalkynyl. Q can be attached to X through a covalent bond to the main portion of X or through a covalent bond to a substitution group of X.

In certain examples of Compound II, it is useful to consider A and X as a single unit. A and X taken together (i.e., A-X) can be, for example, an amino acid. The side chains of acidic and basic amino acids can be protonated or deprotonated. For example, if A is

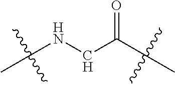

and X is —(CH₂)₂CO₂—, then A-X combined form deprotonated glutamic acid. An example of Compound II where A-X is glutamic acid is as follows:

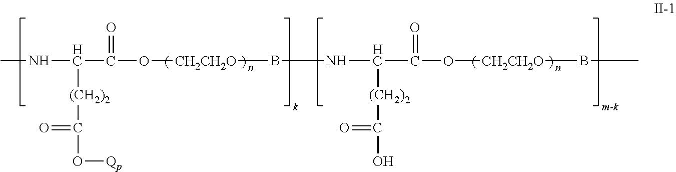

Other naturally and non-naturally occurring amino acids, e.g., arginine; asparagine; aspartic acid; citrulline; cysteine; glutamine; histidine; lanthionine; lysine; ornithine; serine; threonine; tryptophan; and tyrosine, are also useful.

Also in Compound II, m can be an integer of 1 or greater. For example, m can be 1 or greater, 2 or greater, 3 or greater, 4 or greater, 5 or greater, 6 or greater, 7 or greater, 8 or greater, 9 or greater, 10 or greater, 15 or greater, 20 or greater, 25 or greater, 30 or greater, 40 or greater, 50 or greater, 60 or greater, 70 or greater, 80 or greater, 90 or greater, 100 or greater, 125 or greater, 150 or greater, 175 or greater, 200 or greater, 225 or greater, 250 or greater, 275 or greater, 300 or greater, 400 or greater, 500 or greater, 600 or greater, 700 or greater, 800 or greater, 900 or greater, or 1000 or greater. Useful ranges for m include from 1 to 1000, from 1 to 100, from 1 to 10, or subranges thereof.

Additionally in Compound II, n can be an integer of 1 or greater. For example, n can be 1 or greater, 2 or greater, 3 or greater, 4 or greater, 5 or greater, 6 or greater, 7 or greater, 8 or greater, 9 or greater, 10 or greater, 15 or greater, 20 or greater, 25 or greater, 30 or greater, 40 or greater, 50 or greater, 60 or greater, 70 or greater, 80 or greater, 90 or greater, or 100 or greater. Useful ranges for n include from 1 to 100, from 1 to 50, from 1 to 10, or subranges thereof.

Further in Compound II, y¹ and y² can each independently be integers of 1 or greater. For example, y¹ or y² can be 1 or greater, 2 or greater, 3 or greater, 4 or greater, 5 or greater, 6 or greater, 7 or greater, 8 or greater, 9 or greater, or 10 or greater. Useful y¹ and y² values include 1, 2, 3, 4, 5, 6, 7, 8, 9, and 10.

Additionally in Compound II, p can be an integer of 1 or greater. Useful p values include 1, 2, 3, 4, 5, 6, 7, 8, 9, or 10.

Further in Compound II, k can be an integer from 1 to m.

Additional examples of Compound II include:

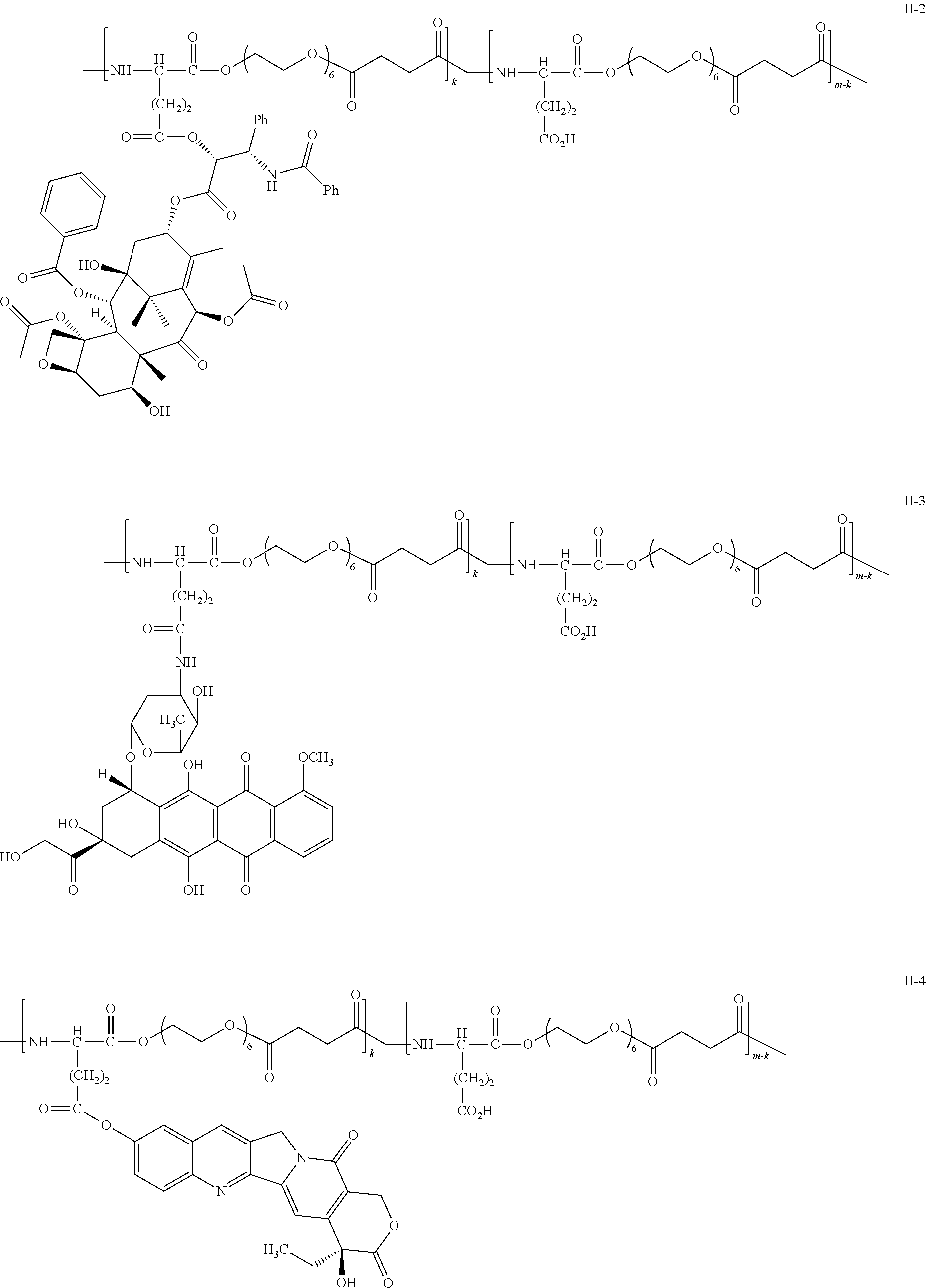

Further described is a method of detecting specific target cells in a subject, comprising administering to the subject an effective amount of a compound as represented by Compound III:

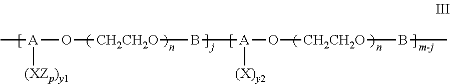

or a pharmaceutically acceptable salt thereof, and detecting localization of the compound by a diagnostic imaging technique. Examples of diagnostic imaging techniques useful with the compounds and methods described herein include magnetic resonance imaging (MRI), computed axial tomography scans (CAT scans or CT scans), and scintigraphy.

In Compound III, A is substituted or unsubstituted C₁₋₁₂ alkyl, substituted or unsubstituted C₂₋₁₂ alkenyl, substituted or unsubstituted C₂₋₁₂ alkynyl, substituted or unsubstituted C₂₋₁₂ heteroalkyl, substituted or unsubstituted C₂₋₁₂ heteroalkenyl, or substituted or unsubstituted C₂₋₁₂ heteroalkynyl.

Also in Compound III, B is substituted or unsubstituted C₁₋₁₂ alkyl, substituted or unsubstituted C₂₋₁₂ alkenyl, substituted or unsubstituted C₂₋₁₂ alkynyl, substituted or unsubstituted C₂₋₁₂ heteroalkyl, substituted or unsubstituted C₂₋₁₂ heteroalkenyl, substituted or unsubstituted C₂₋₁₂ heteroalkynyl, or is absent.

Also in Compound III, m, n, p, y¹, and y² are each independently selected from an integer of one or greater and j is an integer from one to m. As used in Compound III, the overall number of repeat units, i.e., the heterofunctional segment-poly(ethylene glycol) polymer units, is m, and the number of heterofunctional segment-poly(ethylene glycol) polymer units with an attached Z is j. Thus, the number of heterofunctional segment-poly(ethylene glycol) polymer units with no Z is m−j. In one example, m is 45, n is 6, p is 1, y¹ is 1, y² is 1, and j is m (i.e., each heterofunctional segment-poly(ethylene glycol) polymer unit has an attached Z).

Additionally in Compound III, Z is an imaging agent as described herein. The j and m−j sections of Compound III are included because an imaging agent does not necessarily need to be attached to each repeat unit of Compound III for the compound to be effective. Thus, the amount of Z administered to a subject using Compound III will depend on the amount of the imaging agent (Z) delivered by Compound III to the area of interest and subsequently how much Z is imageable in the area of interest.

The orientation of the j and m−j sections of Compound III is not intended to indicate the Z bound subunits are necessarily in a block, but rather that a varying number of Z bound units, i.e., j, is possible. Expressed as a percentage, j can be as little as 1-2% of the total m units or as much as 100% (e.g., m=j). For example, j can be from 1% to 99%, from 5% to 95%, from 10% to 90%, from 20% to 80%, from 30% to 70%, or from 40% to 60% of m. Z can be, for example, a tracer, an imaging atom, or an imaging molecule. Examples of imaging agents useful with the compounds and methods as described herein include MRI contrast-enhancing agents, such as gadoteridol, gadolinium-diethylenetriaminepentaacetic acid complex, and mangafodipir trisodium.

Further in Compound III, X is a linker molecule covalently attached to A. In some examples, X is substituted or unsubstituted C₁₋₁₂ alkyl, substituted or unsubstituted C₂₋₁₂ alkenyl, substituted or unsubstituted C₂₋₁₂ alkynyl, substituted or unsubstituted C₂₋₁₂ heteroalkyl, substituted or unsubstituted C₂₋₁₂ heteroalkenyl, or substituted or unsubstituted C₂₋₁₂ heteroalkynyl. Z can be attached to X through a covalent bond to the main portion of X or through a covalent bond to a substitution group of X.

In certain examples of Compound III, it is useful to consider A and X as a single unit. A and X taken together (i.e., A-X) can be, for example, an amino acid. The side chains of acidic and basic amino acids can be protonated or deprotonated. For example, if A is

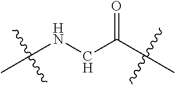

and X is —(CH₂)₂CO₂—, then A-X combined form deprotonated glutamic acid. An example of Compound III where A-X is glutamic acid is as follows:

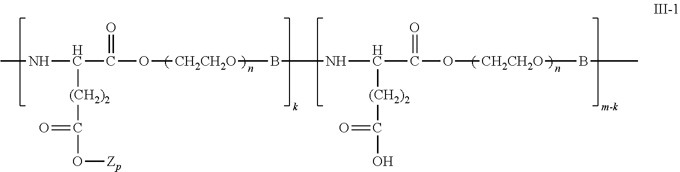

Other naturally and non-naturally occurring amino acids, e.g., arginine; asparagine; aspartic acid; citrulline; cysteine; glutamine; histidine; lanthionine; lysine; ornithine; serine; threonine; tryptophan; and tyrosine, are also useful.

Also in Compound III, m can be an integer of 1 or greater. For example, m can be 1 or greater, 2 or greater, 3 or greater, 4 or greater, 5 or greater, 6 or greater, 7 or greater, 8 or greater, 9 or greater, 10 or greater, 15 or greater, 20 or greater, 25 or greater, 30 or greater, 40 or greater, 50 or greater, 60 or greater, 70 or greater, 80 or greater, 90 or greater, 100 or greater, 125 or greater, 150 or greater, 175 or greater, 200 or greater, 225 or greater, 250 or greater, 275 or greater, 300 or greater, 400 or greater, 500 or greater, 600 or greater, 700 or greater, 800 or greater, 900 or greater, or 1000 or greater. Useful ranges for m include from 1 to 1000, from 1 to 100, from 1 to 10, or subranges thereof.

Additionally in Compound III, n can be an integer of 1 or greater. For example, n can be 1 or greater, 2 or greater, 3 or greater, 4 or greater, 5 or greater, 6 or greater, 7 or greater, 8 or greater, 9 or greater, 10 or greater, 15 or greater, 20 or greater, 25 or greater, 30 or greater, 40 or greater, 50 or greater, 60 or greater, 70 or greater, 80 or greater, 90 or greater, or 100 or greater. Useful ranges for n include from 1 to 100, from 1 to 50, from 1 to 10, or subranges thereof.

Further in Compound III, y¹ and y² can each independently be integers of 1 or greater. For example, y¹ or y² can be 1 or greater, 2 or greater, 3 or greater, 4 or greater, 5 or greater, 6 or greater, 7 or greater, 8 or greater, 9 or greater, or 10 or greater. Useful y¹ and y² values include 1, 2, 3, 4, 5, 6, 7, 8, 9, and 10.

Additionally in Compound III, p can be an integer of 1 or greater. Useful p values include 1, 2, 3, 4, 5, 6, 7, 8, 9, or 10.

Further in Compound III, j can be an integer from 1 to m.

Also described is a method of treating a subject and detecting specific target cells in a subject, comprising administering to the subject an effective amount of a compound as represented by Compound IV:

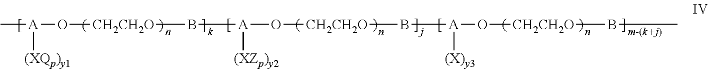

or a pharmaceutically acceptable salt thereof, and detecting localization of the compound by a diagnostic imaging technique. Examples of diagnostic imaging techniques useful with the compounds and methods described herein include magnetic resonance imaging (MRI), computed axial tomography scans (CAT scans or CT scans), and scintigraphy.

In Compound IV, A is substituted or unsubstituted C₁₋₁₂ alkyl, substituted or unsubstituted C₂₋₁₂ alkenyl, substituted or unsubstituted C₂₋₁₂ alkynyl, substituted or unsubstituted C₂₋₁₂ heteroalkyl, substituted or unsubstituted C₂₋₁₂ heteroalkenyl, or substituted or unsubstituted C₂₋₁₂ heteroalkynyl.

Also in Compound IV, B is substituted or unsubstituted C₁₋₁₂ alkyl, substituted or unsubstituted C₂₋₁₂ alkenyl, substituted or unsubstituted C₂₋₁₂ alkynyl, substituted or unsubstituted C₂₋₁₂ heteroalkyl, substituted or unsubstituted C₂₋₁₂ heteroalkenyl, substituted or unsubstituted C₂₋₁₂ heteroalkynyl, or is absent.

Also in Compound IV, m, n, p, y¹, y², and y³ are each independently selected from an integer of one or greater and j and k are each independently selected from an integer from one to m. As used in Compound IV, the overall number of repeat units, i.e., the heterofunctional segment-poly(ethylene glycol) polymer units, is m; the number of heterofunctional segment-poly(ethylene glycol) polymer units with an attached Q is k; and the number of heterofunctional segment-poly(ethylene glycol) polymer units with an attached Z is j. Thus, the number of heterofunctional segment-poly(ethylene glycol) polymer units with no Q or Z is m−(k+j). In one example, m is 45, n is 6, p is 1, y¹ is 1, y² is 1, y³ is 1, k is ⅓ m, and j is ⅔ m (i.e., 15 heterofunctional segment-poly(ethylene glycol) polymer units have an attached Q and 30 heterofunctional segment-poly(ethylene glycol) polymer units have an attached Z).

Additionally in Compound IV, Q is a therapeutic agent and Z is an imaging agent as described herein. The k, j, and m−(k+j) sections of Compound IV are included because a therapeutic agent or imaging agent does not necessarily need to be attached to each repeat unit of Compound IV for the compound to be effective. Thus, the amount of Q and Z administered to a subject using Compound IV will depend on the amount of the therapeutic agent (Q) and the imaging agent (Z) delivered by Compound IV to the area of interest and subsequently how much Q and Z are available in the area of interest.

The orientation of the k, j, and m−(k+j) sections of Compound IV is not intended to indicate the Q bound subunits and Z bound subunits are necessarily in a block, but rather that a varying number of Q bound subunits (i.e., k) and Z bound units (i.e., j) is possible. Examples of therapeutic agents (Q) useful with the compounds and methods as described herein include anti-cancer agents, radioactive isotopes, polypeptides, and carbohydrates. Q can be, for example, paclitaxel, camptothecin, doxorubicin, curcumin, an antibody, or a platinum-derived agent. Z can be, for example, a tracer, an imaging atom, or an imaging molecule. An example of an imaging agent useful with the compounds and methods as described herein includes MRI contrast-enhancing agents.

Further in Compound IV, X is a linker molecule covalently attached to A. In some examples, X is substituted or unsubstituted C₁₋₁₂ alkyl, substituted or unsubstituted C₂₋₁₂ alkenyl, substituted or unsubstituted C₂₋₁₂ alkynyl, substituted or unsubstituted C₂₋₁₂ heteroalkyl, substituted or unsubstituted C₂₋₁₂ heteroalkenyl, or substituted or unsubstituted C₂₋₁₂ heteroalkynyl. Z or Q can be attached to X through a covalent bond to the main portion of X or through a covalent bond to a substitution group of X.

In certain examples of Compound IV, it is useful to consider A and X as a single unit. A and X taken together (i.e., A-X) can be, for example, an amino acid. The side chains of acidic and basic amino acids can be protonated or deprotonated. For example, if A is

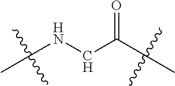

and X is —(CH₂)₂CO₂—, then A-X combined form deprotonated glutamic acid. Other naturally and non-naturally occurring amino acids, e.g., arginine; asparagine; aspartic acid; citrulline; cysteine; glutamine; histidine; lanthionine; lysine; ornithine; serine; threonine; tryptophan; and tyrosine, are also useful.

Also in Compound IV, m can be an integer of 1 or greater. For example, m can be 1 or greater, 2 or greater, 3 or greater, 4 or greater, 5 or greater, 6 or greater, 7 or greater, 8 or greater, 9 or greater, 10 or greater, 15 or greater, 20 or greater, 25 or greater, 30 or greater, 40 or greater, 50 or greater, 60 or greater, 70 or greater, 80 or greater, 90 or greater, 100 or greater, 125 or greater, 150 or greater, 175 or greater, 200 or greater, 225 or greater, 250 or greater, 275 or greater, 300 or greater, 400 or greater, 500 or greater, 600 or greater, 700 or greater, 800 or greater, 900 or greater, or 1000 or greater. Useful ranges for m include from 1 to 1000, from 1 to 100, from 1 to 10, or subranges thereof.

Additionally in Compound IV, n can be an integer of 1 or greater. For example, n can be 1 or greater, 2 or greater, 3 or greater, 4 or greater, 5 or greater, 6 or greater, 7 or greater, 8 or greater, 9 or greater, 10 or greater, 15 or greater, 20 or greater, 25 or greater, 30 or greater, 40 or greater, 50 or greater, 60 or greater, 70 or greater, 80 or greater, 90 or greater, or 100 or greater. Useful ranges for n include from 1 to 100, from 1 to 50, from 1 to 10, or subranges thereof.

Further in Compound IV, y¹, y², and y³ can each independently be integers of 1 or greater. For example, y¹, y², y³ can be 1 or greater, 2 or greater, 3 or greater, 4 or greater, 5 or greater, 6 or greater, 7 or greater, 8 or greater, 9 or greater, or 10 or greater. Useful y¹, y², and y³ values include 1, 2, 3, 4, 5, 6, 7, 8, 9, and 10.

Additionally in Compound IV, p can be an integer of 1 or greater. Useful p values include 1, 2, 3, 4, 5, 6, 7, 8, 9, or 10.

Further in Compound IV, k and j can be integers from 1 to m.

The compounds described herein can be prepared in a variety of ways. The compounds can be synthesized using synthetic methods known in the art of synthetic organic chemistry or variations thereon. The compounds described herein can be prepared from readily available starting materials. Optimum reaction conditions may vary with the particular reactants or solvent used, but such conditions can be determined by one skilled in the art by routine optimization procedures.

Variations on Compound I, Compound II, Compound III, and Compound IV include the addition, subtraction, or movement of the various constituents as described for each compound. Similarly, when one or more chiral centers is present in a molecule the chirality of the molecule can be changed. Additionally, compound synthesis can involve the protection and deprotection of various chemical groups. The use of protection and deprotection, and the selection of appropriate protecting groups can be selected by one skilled in the art. The chemistry of protecting groups can be found, for example, in Wuts and Greene, Protective Groups in Organic Synthesis, 4^{th} Ed., Wiley & Sons, 2006, which is incorporated herein by reference in its entirety.

Reactions to produce the compounds described herein can be carried out in solvents which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography.

An example of a method for making a heterofunctional segment-poly(ethylene glycol) polymer of Compounds I, II, III, and IV is described by Scheme 1.

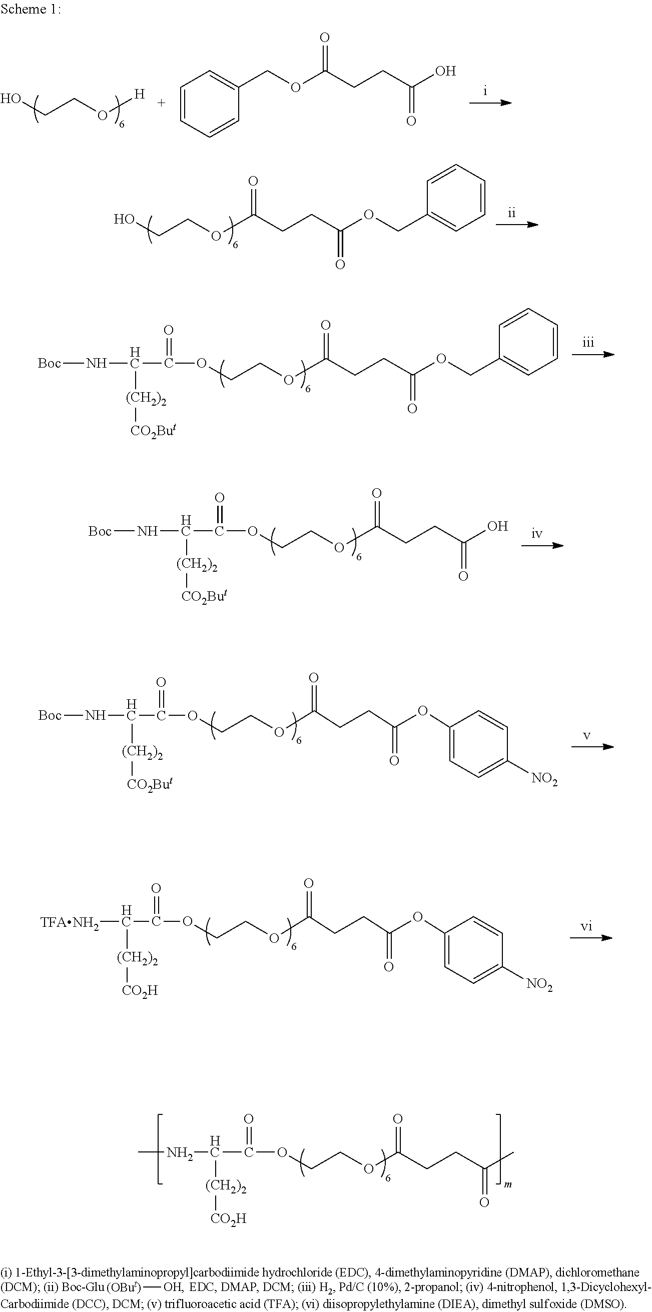

Also provided herein is a method of making a heterofunctional segment-poly(ethylene glycol) polymer of Compounds I, II, and IV comprising conjugating a therapeutic agent to Compound I using a coupling reagent. An example of a method for conjugating a therapeutic agent to the heterofunctional segment-poly(ethylene glycol) polymer of Compounds I, II, and IV is described in Scheme 2.

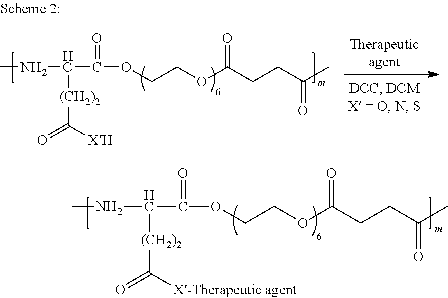

Further provided herein is a method of making a heterofunctional segment-poly(ethylene glycol) polymer of Compounds I, III, and IV comprising conjugating an imaging agent to Compound I using a coupling reagent. An example of a method for conjugating an imaging agent to the heterofunctional segment-poly(ethylene glycol) polymer of Compounds I, III, and IV is described in Scheme 3.

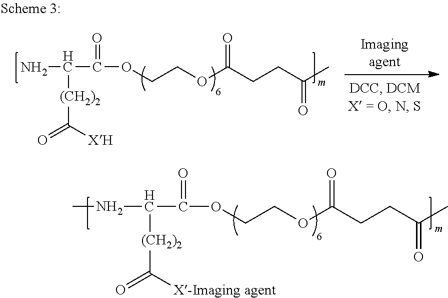

For further example, conjugates of paclitaxel, doxorubicin, and camptothecin can be prepared by the method of Scheme 2, i.e., using carbodiimide coupling reactions. In these reactions, the 2′- and 10-hydroxyl groups of paclitaxel and camptothecin, respectively, and the amine function of doxorubicin, can be coupled to the glutamic acids of a polymer as described herein by adding a coupling reagent such as dicyclohexyl carbodiimide (DCC) to a solution of the reactants in dry dichloromethane. Conjugates of imaging agents can be prepared by the method of Scheme 3, i.e., using carbodiimide coupling reactions.

The compounds described herein or pharmaceutically acceptable salts thereof can be provided in a pharmaceutical composition. Depending on the intended mode of administration, the pharmaceutical composition can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include an effective amount of the compounds described herein or a pharmaceutically acceptable salt thereof in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected substrate without causing significant undesirable biological effects or interacting in a deleterious manner with any of the other components of the pharmaceutical composition in which it is contained.

As used herein, the term carrier encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, University of the Sciences in Philadelphia, Lippincott, Williams & Wilkins, Philadelphia Pa., 2005. Examples of physiologically acceptable carriers include buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN® (ICI, Inc.; Bridgewater, N.J.), polyethylene glycol (PEG), and PLURONICS™ (BASF; Florham Park, N.J.).

Compositions containing one or more of the compounds described herein or pharmaceutically acceptable salts thereof suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example, sugars, sodium chloride, and the like. Prolonged absorption of the injectable pharmaceutical form can be brought about by the use of agents delaying absorption, for example, aluminum monostearate and gelatin.

Solid dosage forms for oral administration of the compounds described herein or a pharmaceutically acceptable salt thereof include capsules, tablets, pills, powders, and granules. In such solid dosage forms, the compounds described herein or a pharmaceutically acceptable salt thereof is admixed with at least one inert customary excipient (or carrier) such as sodium citrate or dicalcium phosphate or (a) fillers or extenders, as for example, starches, lactose, sucrose, glucose, mannitol, and silicic acid; (b) binders, as for example, carboxymethylcellulose, alignates, gelatin, polyvinylpyrrolidone, sucrose, and acacia; (c) humectants, as for example, glycerol; (d) disintegrating agents, as for example, agar-agar, calcium carbonate, potato or tapioca starch, alginic acid, certain complex silicates, and sodium carbonate; (e) solution retarders, as for example, paraffin; (f) absorption accelerators, as for example, quaternary ammonium compounds; (g) wetting agents, as for example, cetyl alcohol, and glycerol monostearate; (h) adsorbents, as for example, kaolin and bentonite; and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others well known in the art. They may contain opacifying agents, and can also be of such composition that they release the active compound or compounds in a certain part of the intestinal tract in a delayed manner. Examples of embedding compositions which can be used are polymeric substances and waxes. The active compounds can also be in micro-encapsulated form, if appropriate, with one or more of the above-mentioned excipients.

Liquid dosage forms for oral administration of the compounds described herein or pharmaceutically acceptable salts thereof include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers. Suitable solvents, solubilizing agents, and emulsifiers include, for example, ethyl alcohol; isopropyl alcohol; ethyl carbonate; ethyl acetate; benzyl alcohol; benzyl alcohol; benzyl benzoate, propyleneglycol; 1,3-butyleneglycol; dimethylformamide; oils, in particular, cottonseed oil, groundnut oil, corn germ oil, olive oil, castor oil, sesame oil; glycerol; tetrahydrofurfuryl alcohol; polyethyleneglycols; and fatty acid esters of sorbitan, or mixtures of these substances, and the like.

Besides such inert diluents, the composition can also include adjuvants, such as wetting, emulsifying, suspending, sweetening, flavoring, or perfuming agents.

Suspensions, in addition to the active compounds, may contain suspending agents, as for example, ethoxylated isostearyl alcohols, polyoxyethylene sorbitol and sorbitan esters; microcrystalline cellulose; aluminum metahydroxide; bentonite; agar-agar; tragacanth; mixtures of these substances; and the like.

Compositions of the compounds described herein or pharmaceutically acceptable salts thereof for rectal administrations are optionally suppositories, which can be prepared by mixing the compounds with suitable non-irritating excipients or carriers such as cocoa butter, polyethyleneglycol or a suppository wax, which are solid at ordinary temperatures but liquid at body temperature and, therefore, melt in the rectum or vaginal cavity and release the active component.

Dosage forms for topical administration of the compounds described herein or pharmaceutically acceptable salts thereof include ointments, powders, sprays, and inhalants. The compounds described herein or pharmaceutically acceptable salts thereof are admixed under sterile conditions with a physiologically acceptable carrier and any preservatives, buffers, or propellants as may be required. Ophthalmic formulations, ointments, powders, and solutions are also contemplated as being within the scope of the compositions.

The term pharmaceutically acceptable salt as used herein refers to those salts of the compounds described herein that are, within the scope of sound medical judgment, suitable for use in contact with the tissues of subjects without undue toxicity, irritation, allergic response, and the like, commensurate with a reasonable benefit/risk ratio, and effective for their intended use, as well as the zwitterionic forms, where possible, of the compounds described herein. The term salts refers to the relatively non-toxic, inorganic and organic acid salts of the compounds described herein. These salts can be prepared in situ during the isolation and purification of the compounds or by separately reacting the purified compound in its free base form with a suitable organic or inorganic acid and isolating the salt thus formed. Representative salts include hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, methane sulphonate, and laurylsulphonate salts, and the like. These may include cations based on alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See Stahl and Wermuth, Pharmaceutical Salts: Properties, Selection, and Use, Wiley—VCH, 2008, which is incorporated herein by reference in its entirety, at least, for compositions taught therein.)

Administration of compounds described herein or pharmaceutically acceptable salts thereof can be carried out using therapeutically effective amounts of the compounds described herein or pharmaceutically acceptable salts thereof. The effective amount of the compounds described herein will depend upon the effective amount of the therapeutic agent being delivered which in turn will depend upon the amount of the therapeutic agent delivered to an area of interest and the availability of the therapeutic agent once delivered. The effective amount of the compounds described herein or pharmaceutically acceptable salts thereof may be determined by one of ordinary skill in the art, and includes exemplary dosage amounts for a mammal of from about 0.05 to about 100 mg/kg of body weight of compound per day, which may be administered in a single dose or in the form of individual divided doses, such as from 1 to 4 times per day. Alternatively, the dosage amount can be from about 0.05 to about 75 mg/kg of body weight of compound per day, about 0.5 to about 50 mg/kg of body weight of compound per day, about 0.5 to about 25 mg/kg of body weight of compound per day, about 1 to about 20 mg/kg of body weight of compound per day, about 1 to about 10 mg/kg of body weight of compound per day, about 20 mg/kg of body weight of compound per day, about 10 mg/kg of body weight of compound per day, or about 5 mg/kg of body weight of compound per day. The specific dose level and frequency of dosage for any particular subject may be varied and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition.

In the methods described herein, the subjects treated can be further treated with one or more additional agents. The one or more additional agents and the compounds described herein or a pharmaceutically acceptable salt thereof can be administered in any order, including simultaneous administration, as well as temporally spaced order of up to several days apart. The methods may also include more than a single administration of the one or more additional agents and/or the compounds described herein or a pharmaceutically acceptable salt thereof. The administration of the one or more additional agents and one or more compounds described herein or pharmaceutically acceptable salts thereof may be by the same or different routes and concurrently or sequentially.

The examples below are intended to further illustrate certain aspects of the methods and compounds described herein, and are not intended to limit the scope of the claims.

### EXAMPLES

The compounds described herein have similar activities to their unbound therapeutic agents in cancer cell models. Specifically, compounds with bound campothecin (e.g., Compound II-4 above) and taxol (e.g., Compound II-2 above) were analyzed alongside unbound campothecin and taxol to compare their activities in human colorectal and prostate cancer cell lines.

The tumor cells were maintained as monolayers in 75-cm² tissue culture flasks using their respective cell culture medium containing 10% fetal bovine serum and 2 mM L-glutamine. The cells were then incubated at 37° C. under a humidified 5% CO₂: air atmosphere (standard conditions) for five days. The cells were harvested in mid-log growth. The cell concentration was then determined using a particle counter (Beckman Coulter, Inc, Fullerton, Calif.). An aliquot of the cell suspension was diluted in culture medium and then delivered to a 24-well tissue culture plate at a range of 10,000 to 30,000 per 1 mL per well. After 24 hours, quadruplicate wells were inoculated with either vehicle (untreated controls) or test drugs at various concentrations. After 24 hours of incubation, the wells were aspirated, washed once with 1 mL PBS, and refilled with 1 mL treatment-free medium. Following a 96-hour incubation under standard conditions from the initial treatment, the viable cells were counted and the numbers were normalized to the percent of untreated controls. The extent of cytotoxicity in treated wells as compared to the controls, and the dose that inhibits 50% cell proliferation (IC₅₀) was calculated for conjugated and unconjugated camptothecin and conjugated and unconjugated paclitaxel using the Microsoft Excel software program (Microsoft Corp., Redmond, Wash.).

As shown in FIG. 1, LS174T (human colorectal cancer) cells were treated with a 5 μM concentration of conjugated campothecin (AS-X-147; i.e., Compound II-4, where [k+(m−k)], i.e., m, is approximately 45 and the molecular weight is approximately 22 kDa) and unconjugated campothecin (CPT-10-OH) in 0.5% DMSO for 24 hours. The activity of the conjugated campothecin was similar to the activity of the unconjugated campothecin.

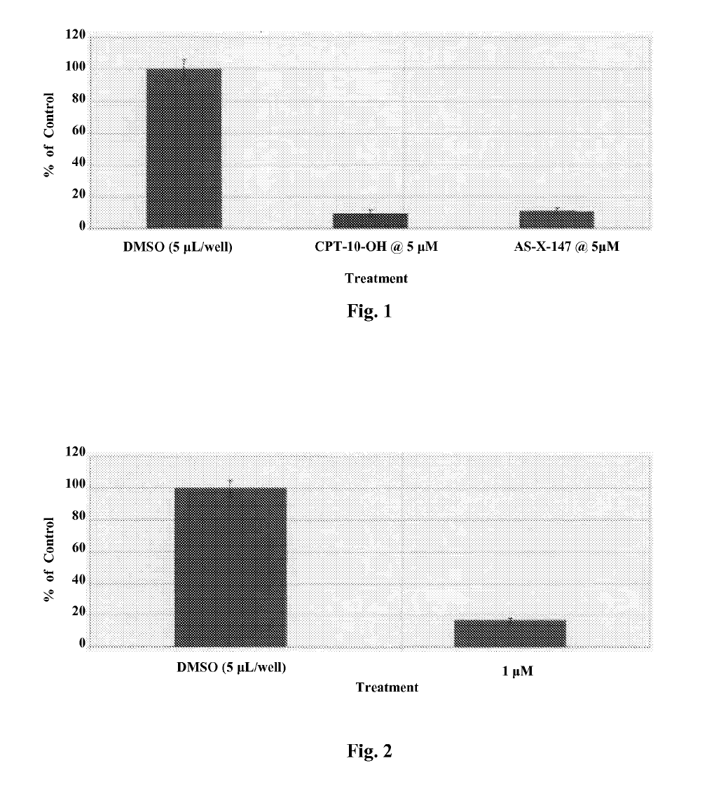

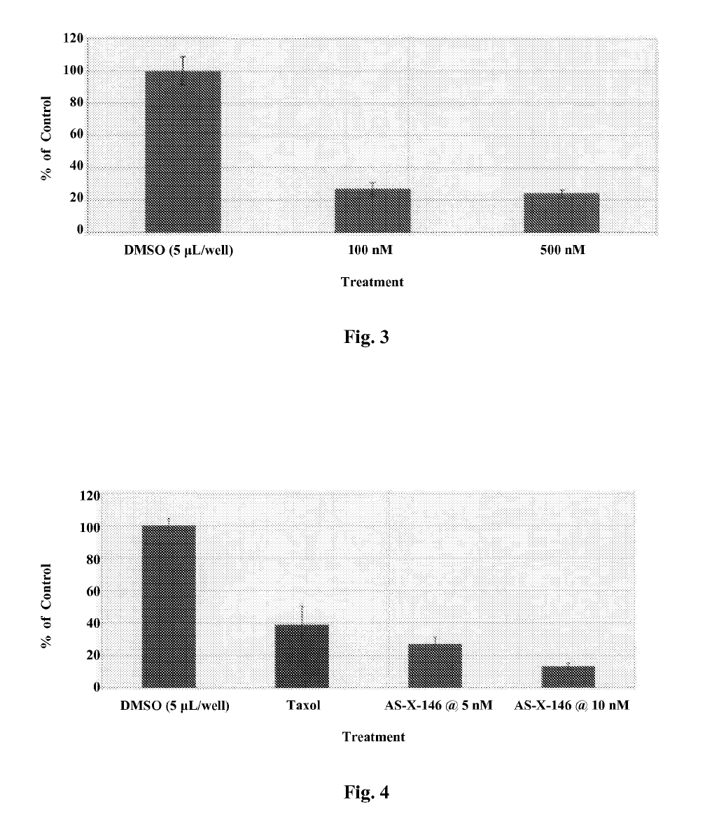

To compare the efficacy of the conjugated campothecin (Compound II-4, where [k+(m−k)], i.e., m, is approximately 45 and the molecular weight is approximately 22 kDa) to unconjugated campothecin, the efficacy of 1 μM concentration of unconjugated campothecin (CPT-10-OH) was first established (see FIG. 2). Next the activity of the conjugated campothecin (AS-X-147) at 100 nM and 500 nM concentrations was evaluated (see FIG. 3). The 100 nM and 500 nM concentrations of conjugated campothecin had similar activities to the 1 μM unconjugated campothecin concentration.

As shown in FIG. 4, PC-3 (prostate cancer) cells were treated with 5 nM and 10 nM concentrations of conjugated (AS-X-146; Compound II-2, where [k+(m−k)], i.e., m, is approximately 45 and the molecular weight is approximately 22 kDa) and unconjugated taxol (Taxol) in 0.5% DMSO for 24 hours. The activity of the conjugated taxol was similar to the activity of the unconjugated taxol.

These examples show that the compounds described herein retain the activity of the therapeutic agent associated with the polymers.

The compounds and methods of the appended claims are not limited in scope by the specific compounds and methods described herein, which are intended as illustrations of a few aspects of the claims and any compounds and methods that are functionally equivalent are within the scope of this disclosure. Various modifications of the compounds and methods in addition to those shown and described herein are intended to fall within the scope of the appended claims. Further, while only certain representative compounds, methods, and aspects of these compounds and methods are specifically described, other compounds and methods are intended to fall within the scope of the appended claims. Thus a combination of steps, elements, components, or constituents may be explicitly mentioned herein; however, all other combinations of steps, elements, components, and constituents are included, even though not explicitly stated.

1. A compound of the following formula: 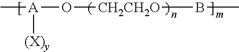
or a salt thereof, wherein:
A is substituted or unsubstituted C₁₋₁₂ alkyl, substituted or unsubstituted C₂₋₁₂ alkenyl, substituted or unsubstituted C₂₋₁₂ alkynyl, substituted or unsubstituted C₂₋₁₂ heteroalkyl, substituted or unsubstituted C₂₋₁₂ heteroalkenyl, or substituted or unsubstituted C₂₋₁₂ heteroalkynyl;
B is substituted or unsubstituted C₁₋₁₂ alkyl, substituted or unsubstituted C₂₋₁₂ alkenyl, substituted or unsubstituted C₂₋₁₂ alkynyl, substituted or unsubstituted C₂₋₁₂ heteroalkyl, substituted or unsubstituted C₂₋₁₂ heteroalkenyl, substituted or unsubstituted C₂₋₁₂ heteroalkynyl, or is absent;
m, n, and y are each independently selected from an integer of one or greater; and
X is a linker molecule covalently attached to A.

2. The compound of claim 1, wherein X is substituted or unsubstituted C₁₋₁₂ alkyl, substituted or unsubstituted C₂₋₁₂ alkenyl, substituted or unsubstituted C₂₋₁₂ alkynyl, substituted or unsubstituted C₂₋₁₂ heteroalkyl, substituted or unsubstituted C₂₋₁₂ heteroalkenyl, or substituted or unsubstituted C₂₋₁₂ heteroalkynyl.

3. The compound of claim 1, wherein A is

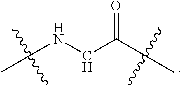

4. The compound of claim 1, wherein A-X is an amino acid.

5. The compound of claim 4, wherein A-X is glutamic acid.

6. The compound of claim 1, wherein n is 6.

7. The compound of claim 1, wherein B is 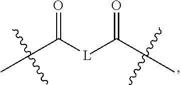
wherein L is a substituted or unsubstituted C₁₋₁₂ alkyl.

8. The compound of claim 7, wherein L is —CH₂—CH₂—.

9. The compound of claim 1, wherein m is 45.

10. The compound of claim 1, wherein one or more of a therapeutic agent and an imaging agent is covalently attached to one or more X.

11. The compound of claim 10, wherein the therapeutic agent is an anti-cancer agent.

12. The compound of claim 11, wherein the anti-cancer agent is paclitaxel, camptothecin, doxorubicin, curcumin, an antibody, or a platinum-derived agent.

13. The compound of claim 10, wherein the therapeutic agent is a radioactive isotope, a polypeptide, or a carbohydrate.

14. The compound of claim 10, wherein the imaging agent is a tracer, an imaging atom, or an imaging molecule.

15. The compound of claim 10, wherein a mixture of therapeutic agents and imaging agents are covalently attached to one or more X.

16. (canceled)

17. (canceled)

18. (canceled)

19. A method of treating a subject, comprising administering to the subject an effective amount of a compound of the following formula: 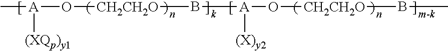
or a pharmaceutically acceptable salt thereof, wherein:
A is substituted or unsubstituted C₁₋₁₂ alkyl, substituted or unsubstituted C₂₋₁₂ alkenyl, substituted or unsubstituted C₂₋₁₂ alkynyl, substituted or unsubstituted C₂₋₁₂ heteroalkyl, substituted or unsubstituted C₂₋₁₂ heteroalkenyl, or substituted or unsubstituted C₂₋₁₂ heteroalkynyl;
B is substituted or unsubstituted C₁₋₁₂ alkyl, substituted or unsubstituted C₂₋₁₂ alkenyl, substituted or unsubstituted C₂₋₁₂ alkynyl, substituted or unsubstituted C₂₋₁₂ heteroalkyl, substituted or unsubstituted C₂₋₁₂ heteroalkenyl, substituted or unsubstituted C₂₋₁₂ heteroalkynyl, or is absent;
m, n, p, y¹, and y² are each independently selected from an integer of one or greater;
k is an integer from one to m;
Q is a therapeutic agent; and
X is a linker molecule covalently attached to A.

20. (canceled)

21. (canceled)

22. (canceled)

23. (canceled)

24. (canceled)

25. (canceled)

26. (canceled)

27. (canceled)

28. The method of claim 19, wherein Q is an anti-cancer agent, a radioactive isotope, a polypeptide, or a carbohydrate.

29. The method of claim 28, wherein the anti-cancer agent is paclitaxel, camptothecin, doxorubicin, curcumin, an antibody, or a platinum-derived agent.

30. (canceled)

31. (canceled)

32. (canceled)

33. (canceled)

34. A method of detecting specific target cells in a subject, comprising administering to the subject an effective amount of a compound of the following formula: 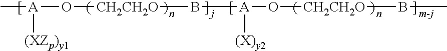
or a pharmaceutically acceptable salt thereof, wherein:
A is substituted or unsubstituted C₁₋₁₂ alkyl, substituted or unsubstituted C₂₋₁₂ alkenyl, substituted or unsubstituted C₂₋₁₂ alkynyl, substituted or unsubstituted C₂₋₁₂ heteroalkyl, substituted or unsubstituted C₂₋₁₂ heteroalkenyl, or substituted or unsubstituted C₂₋₁₂ heteroalkynyl;
B is substituted or unsubstituted C₁₋₁₂ alkyl, substituted or unsubstituted C₂₋₁₂ alkenyl, substituted or unsubstituted C₂₋₁₂ alkynyl, substituted or unsubstituted C₂₋₁₂ heteroalkyl, substituted or unsubstituted C₂₋₁₂ heteroalkynyl, substituted or unsubstituted C₂₋₁₂ heteroalkynyl, or is absent;
m, n, p, y¹, and y² are each independently selected from an integer of one or greater;
j is an integer from one to m;
Z is an imaging agent;
X is a linker molecule covalently attached to A; and
detecting localization of the compound by a diagnostic imaging technique.

35. (canceled)

36. (canceled)

37. (canceled)

38. A method of treating a subject and detecting specific target cells in a subject, comprising administering to the subject an effective amount of a compound of the following formula: 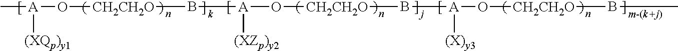
or a pharmaceutically acceptable salt thereof, wherein:
A is substituted or unsubstituted C₁₋₁₂ alkyl, substituted or unsubstituted C₂₋₁₂ alkenyl, substituted or unsubstituted C₂₋₁₂ alkynyl, substituted or unsubstituted C₂₋₁₂ heteroalkyl, substituted or unsubstituted C₂₋₁₂ heteroalkenyl, or substituted or unsubstituted C₂₋₁₂ heteroalkynyl;
B is substituted or unsubstituted C₁₋₁₂ alkyl, substituted or unsubstituted C₂₋₁₂ alkenyl, substituted or unsubstituted C₂₋₁₂ alkynyl, substituted or unsubstituted C₂₋₁₂ heteroalkyl, substituted or unsubstituted C₂₋₁₂ heteroalkenyl, substituted or unsubstituted C₂₋₁₂ heteroalkynyl, or is absent;
m, n, p, y¹, y², and y³ are each independently selected from an integer of one or greater;
j and k are each independently selected from an integer from one to m;
Q is a therapeutic agent;
Z is an imaging agent;
X is a linker molecule covalently attached to A; and
detecting localization of the compound by a diagnostic imaging technique.

39. (canceled)

40. (canceled)

41. (canceled)

42. (canceled)

43. (canceled)

44. (canceled)
